(19) United States
(12) Patent Application Publication
(10) Pub. No.: US 2007/0100466 A1
(43) Pub. Date: May 3, 2007

(54) Prosthetic foot devices

(76) Inventor: Daniel Andrew Allert, Austin, TX (US)

Correspondence Address: MICHAEL O. SCHEINBERG, P.O. BOX 164140, AUSTIN, TX, 78716-4140, US

(21) Appl. No.: 11/525,525

(22) Filed: Sep. 22, 2006

Related U.S. Application Data
(60) Provisional application No. 60/720,433, filed on Sep. 24, 2005.

(51) Int. Cl. A61F 2/66

(52) U.S. Cl. 623/55

(57) ABSTRACT

An improved prosthetic foot achieves minimal weight, robust structure, and anatomically correct behaviors by means of structural arrangement and maximized material application. The improved prosthetic foot includes a forefoot leaf spring longitudinally strengthened by a raised or inverted channel and a heel assembly having a non-linear loading response as the heel member is depressed during a gait cycle. A forefoot according to the invention has a mean flexure line substantially parallel to, and forwardly displaced from, the T_{c} axis of rotation of an equivalent intact foot. The forefoot leaf spring channel provides a hinging action that allows for the expression and isolation of rotational forces at the front of the forefoot (inversion and eversion) separate from the rear of the foot. The hinging action also helps advantageously distribute the pressure of the body weight on the foot in a manner akin to a natural foot. The channel also forms a path for the progression of the center of mass of the body as it progresses through the gait process.

This application claims priority from U.S. Provisional App. No. 60/720,433 filed Sep. 24, 2005, which is hereby incorporated by reference.

### TECHNICAL FIELD OF TEH INVENTION

The present invention relates to a prosthetic foot. More particularly, it relates to an improved prosthetic foot with characteristics of a dynamic response device.

### BACKGROUND AND SUMMARY OF THE INVENTION

Prosthetic feet have undergone major developments in the past several decades, largely spurred by patients demanding full functionality in their prosthesis. Bioengineering research has begun to consider the presence of many complex inter-functionalities in the human form and to address these with a more sophisticated prosthetic design.

There are two general types of current, high-end prosthetic feet: dynamic response and articulating. Dynamic response feet are feet that may be semi-rigid or have a flexible keel, while articulating feet attempt to recreate foot and ankle function.

Popular articulating type prosthetic designs include the Navy ankle, the Greissinger foot, the SACH (Solid Ankle Cushioned Heel) foot, and the Tru-Step™ foot, all of which employ rubber spacers to allow flexure and impact absorption. The benefits of these feet are many, including the fact that they generally have good re-creation of the foot's intact functioning. Unfortunately, their extremely high maintenance requirements and material fatigue make them less than optimal. Additional drawbacks include relatively high weight, complexity of construction, noise resulting from pivoting at bushings, and threat of catastrophic failure.

On the other hand, dynamic type devices are typically lightweight and relatively highly stable. A popular, exemplary conventional dynamic response type foot is known as the Flex-Foot™. It incorporates a flexible carbon fiber shank and heel spring that allows the entire length of the prosthesis (rather than just the foot) to flex, absorb and return energy. Other dynamic response prosthetic feet are currently available with a range of different approaches. Generally using some type of composite (laminated or injection molded) in conjunction with metallic hardware, they are conjoined to an endo-skeletal assembly, which joins the prosthetic foot to the stump socket of the wearer. The carbon beam types are quite popular with users because of their robust and lightweight nature. In some models, the laminated beams may be split down the centerline, allowing for either side of the foot to move relatively independently of the other, providing increased response and stability. Unfortunately, however, the presently available dynamic response type devices lack the flexibility and accurate ankle replication response of the articulating devices. In addition, many of the dynamic devices require a dedicated type of leg shaft.

An improved prosthetic foot is described by the present Applicant in U.S. patent application Ser. No. 10/832,610, entitled “Prosthetic Foot Devices,” which is hereby incorporated by reference. Although the prosthetic foot described by that application is an improvement over the prior art, there is still a need for a more robust prosthetic foot design that weighs less while still providing correct anatomical behavioral characteristics.

### SUMMARY OF THE INVENTION

An object of the invention, therefore, is to provide an improved prosthetic foot. Embodiments of the invention can achieve minimal weight, robust structure, and anatomically correct behaviors by means of structural arrangement and maximized material application. A preferred embodiment uses a minimum of components while allowing for maximum user adjustment through a modular approach to design.

The foregoing has outlined rather broadly the features and technical advantages of the present invention in order that the detailed description of the invention that follows may be better understood. Additional features and advantages of the invention will be described hereinafter. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more thorough understanding of the present invention, and advantages thereof, reference is now made to the following descriptions taken in conjunction with the accompanying drawings, in which:

FIG. 1A shows an exploded view of a preferred embodiment of a prosthetic foot according to the present invention.

FIG. 1B shows an assembled view of the preferred embodiment of FIG. 1A.

FIG. 1C shows a forefoot component from the embodiment of FIG. 1B.

FIG. 2A is a top view of a natural foot showing the talocrural (T_{c}) and talocruronavicular (T_{cn}) axes.

FIG. 2B is a side view of a natural foot showing the talocrural (T_{c}) and talocruronavicular (T_{cn}) axes.

FIG. 3 is a top down view of a forefoot according to the present invention showing the forefoot centerline, center of mass path, and T_{cn} flexure line.

FIG. 4A shows an exploded view of another preferred embodiment of a prosthetic foot according to the present invention.

FIG. 4B shows an assembled view of the preferred embodiment of FIG. 4A.

FIG. 5A shows an exploded view of another preferred embodiment of a prosthetic foot according to the present invention.

FIG. 5B shows an assembled view of the preferred embodiment of FIG. 5A.

FIG. 6A is a graph showing the nonlinear moment of resistance in an intact ankle.

FIG. 6B shows a graph of the moment of resistance versus its angle of deflection for a model of an intact ankle.

### DETAILED DESCRIPTION OF PREFERRED EMBODIMENTS

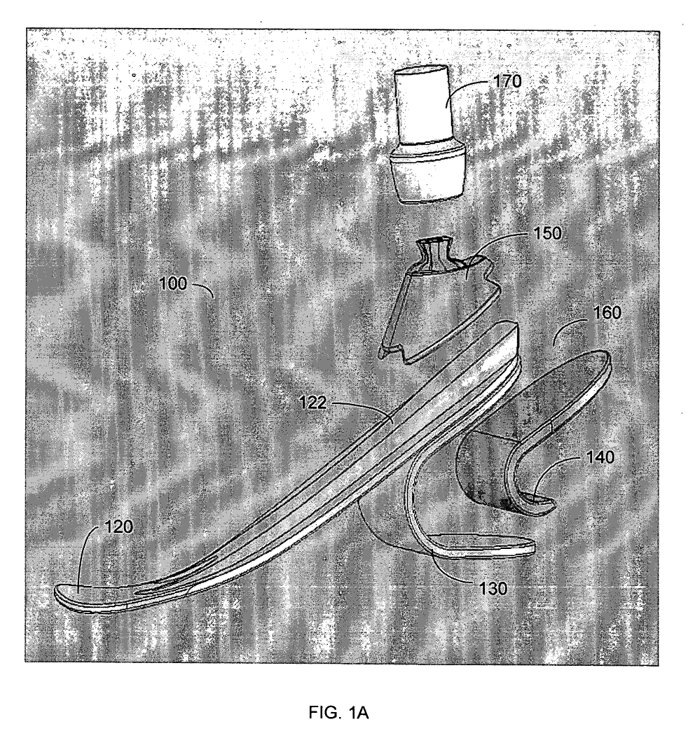

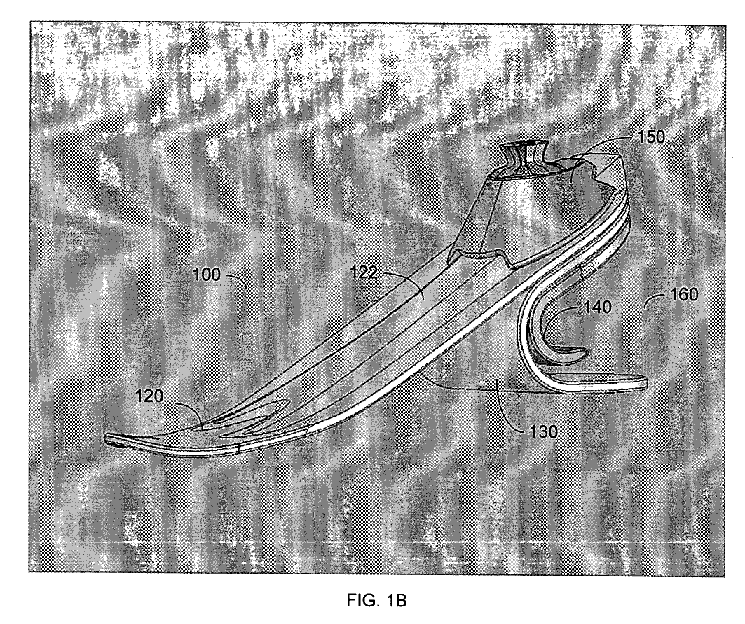

FIG. 1A shows a preferred embodiment of a right side prosthetic foot 100 according to the present invention. (A left side foot is a mirror of the foot 100 and thus will not be discussed.) The foot 100 generally comprises a forefoot 120; a heel assembly 160 with a heel left spring 130 and a heel spring plate 140; and a pyramid adapter mount 150. In the following sections, the forefoot 120, heel leaf spring 130, and heel spring plate 140 will separately be addressed with regard to their structures and operations.

Pyramid adapter mount 150 is preferably composed of titanium metal that may also be embodied in a fiber reinforced, injection molded plastic or a number of other metal or plastic materials. The pyramid adapter mount 150 serves as the intermediary mounting surface between the forefoot leaf spring and a typical industry standard pyramid adapter 170. In the embodiment shown in FIG. 1B, pyramid adapter mount 150 is located on top of the forefoot 120 directly over heel assembly 160. The pyramid adapter mount is preferably completely modular in that a number of iterations could be provided with the prosthetic at different heights, allowing multiple configurations of adapter build height. This allows the user to modify behavioral characteristics of the prosthetic to optimize the foot under foreseeable individual requirements and environmental situations.

Forefoot

The preferred forefoot 120 comprises a leaf spring 121 preferably made of a laminated carbon composite material. Alternatively, the forefoot leaf spring 121 may be composed of a titanium metal or a solid, three-dimensional weave carbon composite. Forefoot 120 is preferably concavely curved toward the limb of the wearer of the prosthetic foot and extends distally (away from the wearer's body) in the same manner as a natural foot. The forefoot 120 is located between the pyramid adapter mount 150 and the heel assembly 160. In the preferred embodiment shown in FIG. 1A to FIG. 1C, raised channel architecture is used to give additional longitudinal support to the forefoot leaf spring without significantly increasing the weight of forefoot 120.

In a preferred embodiment, the raised channel 122 forms an open box structure over much of the length of the forefoot device. In the embodiment shown in FIG. 1A to FIG. 1C, the open side of the open box structure is facing down toward the bottom of the prosthetic foot. As shown in FIG. 1A to FIG. 1C, the channel is located generally in the center of forefoot leaf spring and is raised above the surface of the forefoot leaf spring. Skilled persons will recognize that the center channel need not be located in the exact center of the forefoot. For example, the center channel could be offset toward the outside edge of the forefoot leaf spring so that the inner uninterrupted surface of the forefoot leaf spring is wider than the outer portion. The location of the center channel may be manipulated in order to more closely approximate an individual's particular gait and anatomical variations.

When viewed from the bottom, forefoot 120 would give the appearance of an interrupted platform. As discussed below, an inverted channel could also be used, with the inverted channel below the upper surface of the forefoot leaf spring 121 and the open side facing up, away from the bottom of the prosthetic foot.

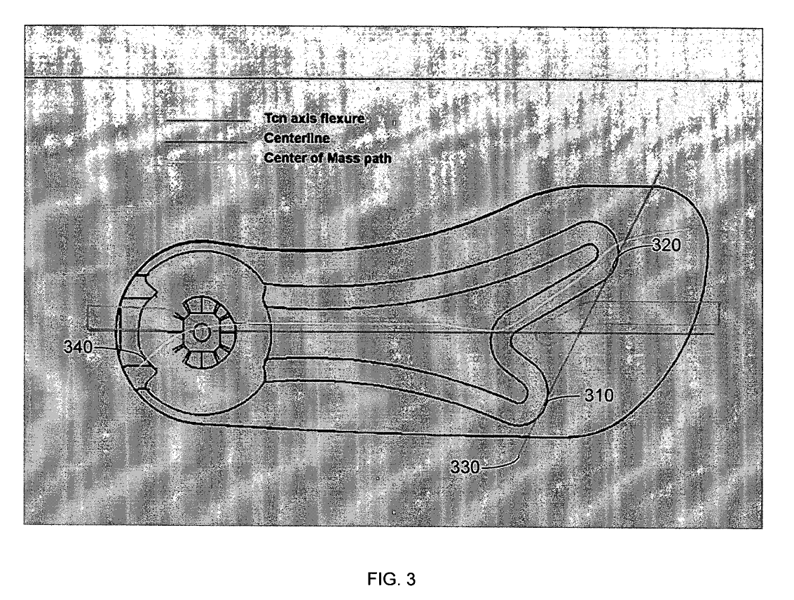

In addition to providing structural support, the open channel also serves as a transverse “hinge,” allowing for the expression and isolation of rotational forces at the front of foot 100 (inversion and eversion) separate from the rear of the foot. This in turn isolates the anatomical stump/socket interface from torque and friction forces magnified along the prosthetic foot's artificial lever. The hinging action also helps advantageously distribute the pressure of the body weight on the foot in a manner akin to a natural foot. As shown in FIG. 3, the channel also forms a path for the progression of the center of mass of the body (shown by line 340) as it progresses through the gait process.

Forefoot 120 uses engineered pivot points in the architecture of the leaf spring to control the location of flexure. As shown in FIG. 1C, the raised channel 122 runs from the proximal end or rear of the forefoot device toward the distal or front of the forefoot device. Because the raised channel serves to give additional longitudinal support, upward force exerted on the forefoot device will not cause the forefoot device to flex along the length where the raised channel is present. The location of termination points 310 and 320, each located toward an outer edge of the forefoot device, determine the location of a flexure line, as indicated by line 330. Because no additional structural support is present in this portion of the forefoot device, upward pressure applied to the forefoot device will cause the device to flex along this mean line of flexure.

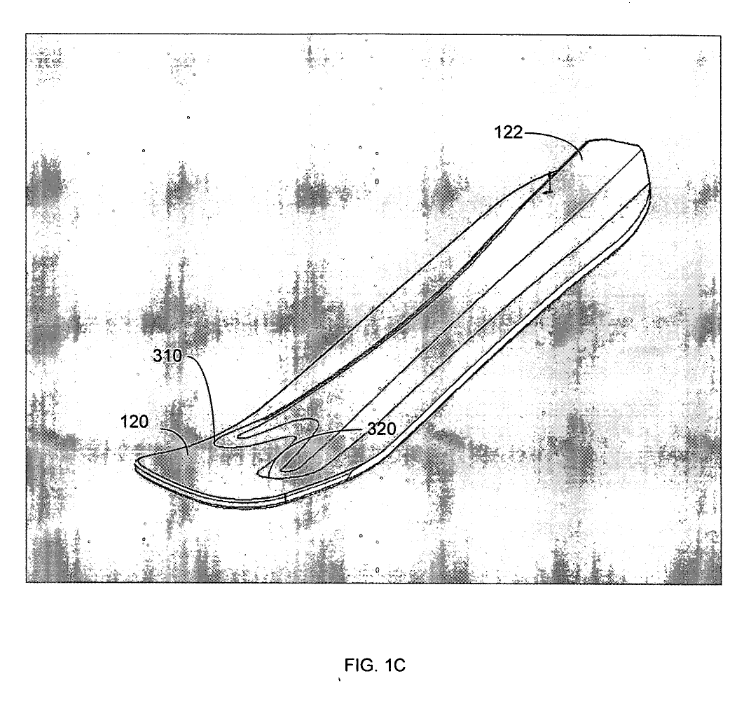

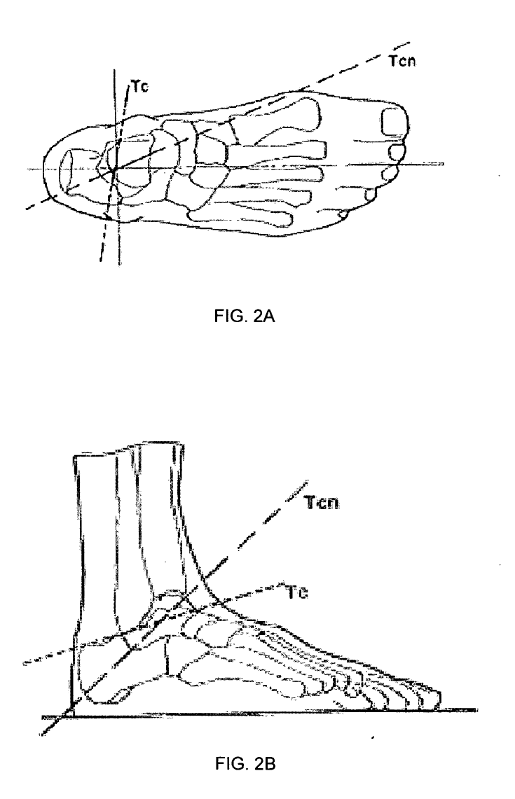

When the mean line of flexure 330 is compared to the T_{c} axis of the natural foot shown in FIG. 2A and FIG. 2B, it is evident that the line 330 is substantially parallel to the natural T_{c} axis but forwardly displaced from it. Shifting the mean line of flexure 330 forward of the natural T_{c} axis in this manner is advantageous as it increases the user's ankle stability while allowing a normal range of motion.

The channel's terminations thus form a flexural location across the forefoot that closely approximate the intact anatomy's talo-crural-navicular (T_{cn}) axis as shown in FIG. 2A and FIG. 2B. This flexural location means that the intact anatomical behavior of the ankle is facilitated without compromising the stability of the rear-foot of the prosthetic. Without the allowance for this biomechanical behavior, other prosthetics experience high stresses upon more traditional mechanical pivots, which lead to component degradation and/or failure, as well as creating unnecessary torquing moments on the stump socket.

In a preferred embodiment of the present invention, the distal termination points for the forefoot channel (whether raised or inverted as discussed below) can be custom tuned for an individual wearer. Depending on an individual's anatomy and preferences, it may be desirable to locate the T_{cn} axis flexure at a particular location. By changing the location of the channel termination points, including changing the location of the termination points relative to each other, the exact location of the T_{cn} axis flexure can be customized for a particular wearer.

The channel 122 may be solid or hollow. While a raised channel is described above, other structures may be employed to provide the same characteristics. Relative to currently available designs, this allows a minimum of material usage and weight while still maximizing robustness.

Heel Assembly

FIG. 1A and FIG. 1B also show a preferred embodiment of a heel leaf spring 130 and a heel spring plate 140 according to the present invention. Together, heel leaf spring 130 and heel spring plate 140 form heel assembly 160.

Heel leaf spring 130 is preferably a resilient leaf spring composed of a laminated carbon composite material that may also be embodied in spring titanium metal or a solid, three-dimensional weave carbon composite. The heel leaf spring 130 is a concavely curved body located in the heel assembly between the forefoot component 120 and the heel spring plate component 140. The heel leaf spring serves as the initial loading structure as the foot enters the heel strike portion of the gait process, absorbing impact forces and storing them for energy return later in the gait process. The heel leaf spring works in combination with the heel spring plate 140 as a progressively loading spring structure.

Heel spring plate 140 is preferably a leaf spring composed of a laminated carbon composite material that may also be embodied in spring titanium metal or a solid, three-dimensional weave carbon composite. The heel spring plate 140 is a concavely curved body located in the heel assembly below the heel leaf spring. The heel spring plate serves as the secondary loading structure as the foot enters the heel strike portion of the gait process, absorbing impact forces and storing them for energy return later in the gait process.

In combination, heel leaf spring 130 and heel spring plate 140 provide a progressive loading response when the lower surface of heel leaf spring 130 comes into contact with the ground. This progressive loading response is due to the shortening of the heel leaf spring 130 as its contact point progresses down the curve of the face of the heel spring plate 140, the changes in effective spring length achieving a desired non-linear load response to the load placed upon it in order to more closely mimic intact anatomical musculo-skeletal arrangements. This causes initial low resistance with increasing resistance as heel strike progresses.

The changes in effective spring length make it easier to achieve a desired non-linear load response to the load that is placed upon it in order to more closely mimic intact anatomical muscular-skeletal arrangements. This is of course desirable. Put another way, the nonlinear loading response of the varying effective spring length of the heel leaf spring 130 causes initial low resistance with increasing resistance as heel strike progresses. The load response, of course, is also a function of the designed spring characteristics of the heel leaf spring itself. For example, if the heel leaf spring is made from a carbon fiber laminate composite, factors such as material type, layer density, and ply orientation can be selected, as known to persons of skill in the art, to provide a heel member with desired spring load characteristics. To a certain extent, the heel leaf spring can be designed to have a non-linear response, but it has been found that a desired response can be more readily attained by controllably shortening its effective length as it is being depressed (as discussed above) in cooperation with the use of a suitable heel spring plate.

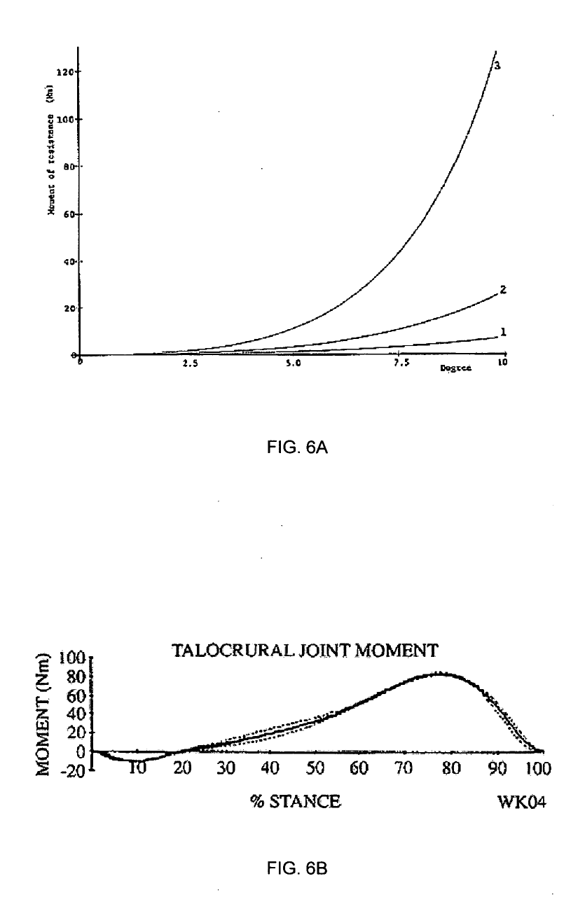

With reference to FIGS. 6A and 6B, in one embodiment, both heel members are designed so that the load response of heel assembly corresponds to the nonlinear moment of resistance in the ankle for an intact person, which is depicted in FIG. 6A. One aid to achieving this is through the use of the graph of FIG. 6B, which shows the moment of resistance versus its angle of deflection for a model of an intact ankle, derived by Dr. Mark Pitkin and described in his article entitled, “Mechanical Outcomes of a Rolling Joint Prosthetic,” American Academy of Orthotists and Prosthetists, Journal of Prosthetics and Orthotics, Vol. 7, No. 4, pp. 114-123 (1995) and its enumerations of the non-linear moment of resistance in the ankle, and the moment of resistance in the ankle versus the angle of deflection. In a preferred embodiment, the non-linear, progressive loading of heel assembly 160 substantially matches the anatomical curve as described by Pitkin.

In a preferred embodiment, heel spring plate 140 also serves to stiffen the overall flexibility of the heel assembly 160 and thus acts as a stabilizing feature when an amputee is standing in place.

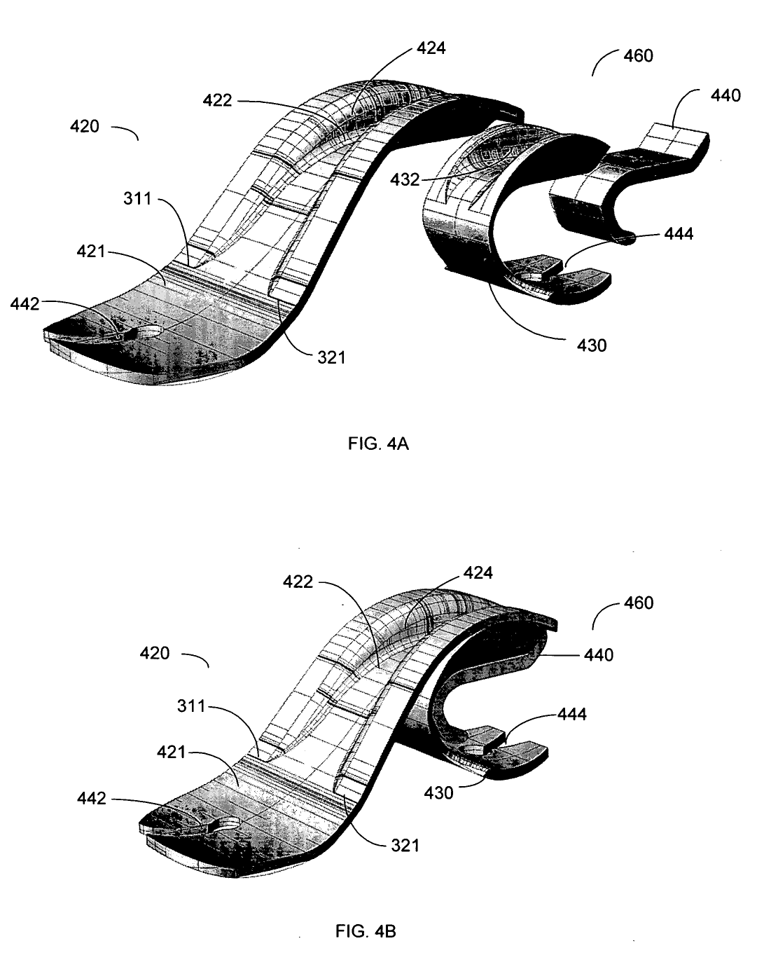

Another preferred embodiment of a prosthetic foot according to the present invention is shown in FIG. 4A and FIG. 4B. In this preferred embodiment, an inverted channel 422 is used instead of the raised channel discussed above. Like the embodiment shown in FIG. 1A to FIG. 1C, channel 422 forms an open box structure over much of the length of the forefoot device. In this embodiment, however, the open area of the open box structure is facing up, away from the bottom of the prosthetic foot, and is bounded by channel sidewalls 424.

The inverted channel of FIG. 4A and FIG. 4B will be more robust than the embodiment shown FIG. 1A to FIG. 1C, while retaining many of the same features and advantages, such as the hinging action discussed above. When the raised channel leaf spring 120 is flexed in a downward direction, the channel walls (not shown) will tend to spread apart. This spreading tends to lessen the longitudinal support provided by the channel and also contributes to laminate shear when the leaf is composed of a laminated carbon composite material. The inverted channel of FIG. 4A and FIG. 4B, however, will not spread when the leaf 422 is bent downward. Instead, the walls of the inverted channel 424 will bow inward, increasing the stiffness and helping to prevent laminate shear. As shown by FIG. 4A and FIG. 4B, the profiles of the heel assembly components 460 (including heel leaf spring 430 and heel spring plate 440) can be adapted to “cup” into the open channel on the bottom of forefoot 420, providing for increased stability and easier assembly. FIG. 4A shows a heel leaf spring 430 that also has an inverted channel 432 formed so that it will mate with the inverted channel of the forefoot.

As in the raised channel embodiment discussed above, forefoot 420 uses engineered pivot points in the architecture of the leaf spring to control the location of flexure. Because the inverted channel also serves to give additional longitudinal support, upward force exerted on the bottom surface of the forefoot device will not cause the forefoot device to flex along the length where the inverted channel is present. Thus, as with the raised channel embodiment discussed above, the location of termination points 311 and 321, each located toward an outer edge of the forefoot device, determine the location of a flexure line. In a preferred embodiment this mean line of flexure will be substantially parallel to the T_{c} axis of a natural foot but forwardly displaced from it.

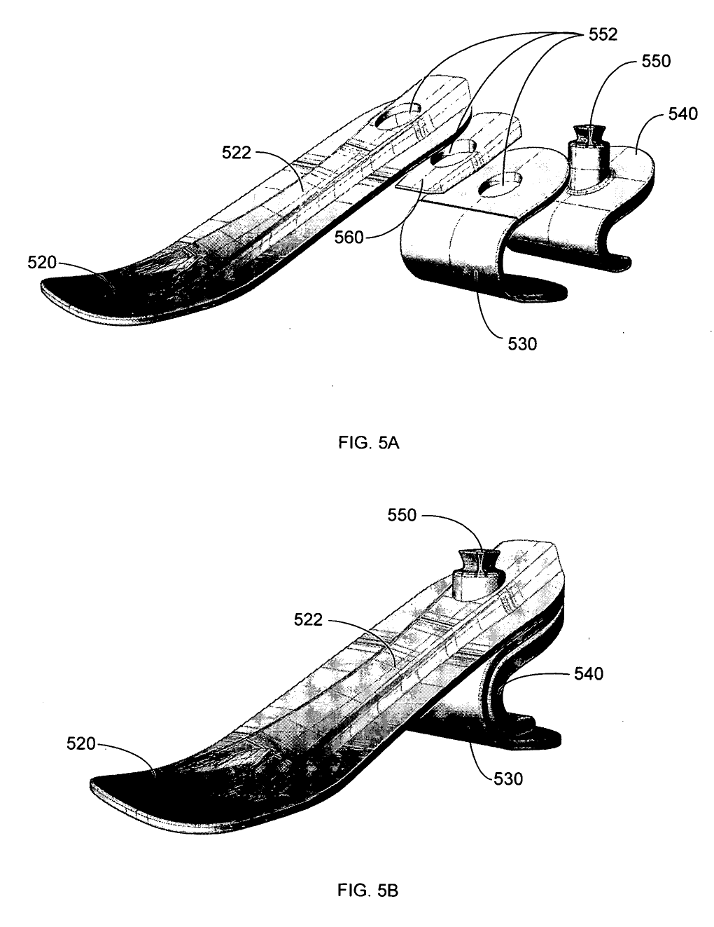

FIGS. 5A and 5B show another preferred embodiment of a prosthetic foot according to the present invention. In the embodiment shown, a raised channel 522 is employed. Instead of a separate pyramid adapter mount located on top of the forefoot, pyramid adapter mount 550 is formed as part of heel spring plate 540. Mounting holes 552 allow the mount to pass through heel leaf spring 530 and forefoot 520 to reach a typical industry standard pyramid adapter (not shown). Spacer 524 is located between forefoot 520 and heel leaf spring 530 and within the open channel 522 on the bottom of forefoot 520. Spacer 524 serves both as a locator for easier assembly of the prosthetic foot assembly and as a support to prevent channel 522 from deforming if the connections between the forefoot and heel assemblies are over-tightened or if the foot is exposed to a large degree of force (as when a wearer jumps down from a higher surface onto a lower surface).

In a preferred embodiment of the present invention, the forefoot leaf spring outboard edge extends below the horizontal plane with respect to the inboard edge, thereby creating an imbalanced contact surface on the forefoot. This acts to force a more natural supinated foot rollover response as the gait process progresses from heel strike into foot flat, as well as providing increased proprioception for the user, avoiding “foot slap” and other undesirable gait behaviors common among prosthetic users.

As shown in FIGS. 4A and 4B, the forefoot leaf spring 421 may also employ a longitudinal split 442 along a pre-determined path which divides the forefoot into two separate “toes,” a “big toe” (on the inboard edge) and a “little toe” (on the outboard edge). These toes serve to re-create the anatomical functions of the first through second tarsal-metatarsal group and the third through fifth tarsal-metatarsal groups, respectively, as the foot progresses from the foot flat into the toe-off portions of the gait process. Heel leaf spring 430 can employ a similar split 444. The divided “toes” can also serve to provide compliance of the foot's contact surface with the ground and any irregularities by providing relatively separate flexure bodies.

Preferred embodiments of the present invention allow for provision of adjustment of relative heel height, thereby allowing the prosthetic foot to be used with a range of shoes. Some embodiments include a “clip-on” type forefoot pad. This forefoot pad is preferably manufactured as a co-molded plastic/rubber structure of multiple iterations at different heights serving to allow multiple configurations of build height of the forefoot relative to the heel, effectively raising the heel height and allowing the use of a wide range of shoes.

This application describes multiple aspects of an improved prosthetic foot. Several of the aspects are thought to be individually novel and not obvious, and separately patentable. Not every embodiment requires every one of these aspects, and the claims are not limited to such an embodiment.

The invention in the preferred embodiments provides correct anatomical behavioral characteristics by progressive loading of the foot components and by relieving torque stresses on the stump and increasing proprioceptive feedback to the user, thus increasing confidence and consequently quality of life through usage. While the structures describe above can provide those advantages, the invention is not limited to the structures described, and alternative structures can also be used.

Although the present invention and its advantages have been described in detail, it should be understood that various changes, substitutions and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim as follows:

1. A prosthetic foot, comprising:
a forefoot having a mean flexure line substantially parallel to, and forwardly displaced from, the T_{c} axis of rotation of an equivalent intact foot; and
a resilient heel member, said heel member having a non-linear loading response as the heel member is depressed during a gait cycle.

2. The prosthetic foot of claim 1 wherein the forefoot further comprises:
a rearward proximal end adapted for mounting within the prosthetic foot;
a forward distal end concavely curved towards a user's limb;
the forefoot having a rearward relatively strengthened non-flexing region such that when said forefoot is loaded, the mean line of flexure of the forefoot is forward of said non-flexing region.

3. The prosthetic foot of claim 1 wherein said strengthened non-flexing region is formed by a center channel.

4. The prosthetic foot of claim 3 wherein said center channel acts as a transverse hinge, allowing for the expression and isolation of rotational forces at said distal end from said proximal end.

5. The prosthetic foot of claim 3 wherein said center channel comprises a raised channel.

6. The prosthetic foot of claim 3 wherein said center channel comprises an inverted channel.

7. The prosthetic foot of claim 2 wherein said resilient heel member is mounted so that the effective spring length of the heel member shortens when the heel member is loaded thereby providing a non-linear loading response as the heel member is being depressed in a gait cycle.

8. A forefoot for a dynamic response prosthetic foot, the forefoot comprising:
a rearward proximal end adapted for mounting within the prosthetic foot;
a forward distal end concavely curved towards a user's limb;
the forefoot having a relatively rearward strengthened non-flexing region, wherein, in use, the mean line of flexure of the forefoot is forward of said non-flexing region.

9. The forefoot of claim 8 wherein the mean line of flexure is substantially parallel to, and forwardly displaced from, the T_{c} axis of rotation of an equivalent intact foot.

10. The forefoot of claim 8 wherein the strengthened non-flexing region is formed by a center channel.

11. The forefoot of claim 10 wherein the mean line of flexure is determined by the forward location of the center channel termination points.

12. The forefoot of claim 10 wherein said center channel provides a path for the progression of the center of mass of a wearer's body as it progresses through the gait process.

13. The forefoot of claim 10 wherein said center channel acts as a transverse hinge, allowing for the expression and isolation of rotational forces at said distal end from said proximal end.

14. The forefoot of claim 8 wherein the proximal end and distal end are integrally formed from a carbon fiber composite material, a laminated carbon composite material, a titanium metal, or a solid, three-dimensional weave carbon composite.

15. The forefoot of claim 8 wherein the forefoot comprises a leaf spring.

16. A heel assembly for a prosthetic foot, the assembly comprising:
a first resilient heel member adapted to be mounted in a prosthetic foot device, said heel member having an effective spring length;
said heel member being mounted so that the effective spring length of the heel member shortens when the heel member is loaded, thereby providing a non-linear loading response as the heel member is being depressed in a gait cycle.

17. The heel assembly of claim 16 wherein said non-linear loading response substantially corresponds to the loading response of an intact ankle.

18. The heel assembly of claim 16 wherein a second heel member mounted so that the second heel member makes contact with a portion of the first heel member when said first member is in a relaxed state; and wherein the second heel member is mounted so that as the first heel member compresses under a load, more of the first heel member makes contact with the second heel member so that the effective spring length of first heel member is shortened as the first heel member is loaded.

19. The heel assembly of claim 18 wherein said first heel member comprises a heel leaf spring and said second heel member comprises a heel spring plate, and wherein said heel leaf spring is mounted to the heel spring plate, the heel leaf spring being formed in the shape of a curve generally convex in shape toward the heel spring plate and the heel spring plate also generally concave in shape in the same direction but having a smaller curve diameter than the heel leaf spring, and the heel leaf spring mounted so that the upper portion of the heel leaf spring is in contact with the heel spring plate when the heel leaf spring is in an unloaded condition.

20. The heel assembly of claim 19 wherein as the heel leaf spring is loaded, the contact between the heal leaf spring and the heel spring plate progresses down the curve of the face of the heel spring plate, the changes in effective spring length achieving a non-linear load response with increasing resistance as the heel leaf spring is loaded during a wearer's gait process.
